(19) United States
(12) Patent Application Publication
(10) Pub. No.: US 2023/0098431 A1
(43) Pub. Date: Mar. 30, 2023

(54) PROCESS FOR HAIR GROWTH

(71) Applicant: ATLANTIC GRACE LLC, New York, NY (US)

(72) Inventor: Kiyomi HORIE, New York, NY (US)

(73) Assignee: ATLANTIC GRACE LLC, New York, NY (US)

(21) Appl. No.: 17/653,960

(22) Filed: Mar. 8, 2022

Related U.S. Application Data
(60) Provisional application No. 63/261,933, filed on Sep. 30, 2021.

(51) Int. Cl. A61M 37/00; A61P 17/14; A61Q 7/00

(52) U.S. Cl. CPC A61M 37/0015; A61P 17/14; A61Q 7/00; A61M 2037/0061

(57) ABSTRACT

A method for treatment for rejuvenation of hair growth combines three phases including a first phase typically applied once a month of micro-needling the area to be treated and thereafter applying an effective amount of a therapeutic; a second phase of applying an electric current and thereafter applying an effective amount of a therapeutic; the first phase typically being applied monthly and a third phase of a mechanical massage; the second and third phases likely being applied twice per week.

### CROSS-REFERENCE TO RELATED APPLICATIONS

This application claims the benefit of priority of U.S. Provisional Application Ser. No. 63/261,933 filed on Sep. 30, 2021, the contents of which are hereby incorporated herein by reference.

### FIELD OF THE PRESENT INVENTION

The present disclosure relates to methods of retarding hair loss or facilitating hair growth and/or regrowth. More specifically, the present invention relates to methods of treatment and use of the disclosed compositions to increase the rate of hair growth.

### BACKGROUND OF THE PRESENT INVENTION

Hair loss, often referred to as Alopecia, in its various forms is an ongoing problem afflicting both men and women, as well as children, and can result from one, or a combination of factors including genetic factors, hormonal factors, surgery, trauma and stress. The universality of the occurrence of alopecia has led to continuing efforts throughout history to discover methods and therapeutic compositions for stimulating hair growth and preventing hair loss.

Numerous remedies for alopecia have been proposed, such as described in Japanese patent application 2012523732A. However, traditional, as well as experimental methods and therapeutic compounds to minimize hair loss or actually stimulate regrowth have typically proved to have very little, if any, effect; or can be rather harmful or unpleasant for the person being treated. Accordingly, an object of the present invention is to provide a minimally invasive method for reducing hair loss, or facilitate hair growth and/or regrowth for those suffering hair loss.

The present invention relates to a process for stimulating the growth of hair, typically on the scalp; but useful in other areas of the human body where hair growth is desired. An important feature of the present invention is an initial step of micro-needling the dermis of the scalp or other area of application and then the application of a therapeutic effective hair growth portion, and thereafter stimulation with an electric current, and subsequently mechanical vibrating massage.

### DETAILED DESCRIPTION OF THE PRESENT INVENTION

The present invention may be described as a treatment method requiring three separate processes; described more fully below in sections I, II and III.

I. First Process Includes Two Steps.

The first step is micro-needling the area of application, typically the dermis of the scalp, and application of a therapeutic effective hair growth serum or portion. The micro-needling of the dermis of the area of application softens the area to create a proper environment to promote hair growth. In view of the use of a micro-needling technique, which slightly penetrates the skin of the area to be treated, the area needs to be properly cleaned and sanitized prior to commencement of the procedure.

A preferred product for the micro-needling process is the Meso TouchPlus manufactured by Merry Laz USA LLC, described at https://merrylaz.com/product/meso-touch-plus/

After the micro-needling, the second step is an application of a therapeutic effective hair growth serum into the micro holes created by the micro-needling process described above. A preferred product to be used is Derma SerumHair Growth supplied by Merry Laz USA LLC described at Website: https://merrylaz.com/product/hair-growth/

This product includes as its ingredients water, butylene glycol, hydrolyzed beta-glucan, palmitoyl tripeptide-1, polyglutamic acid, algae extract, hydrolyzed sclerotium gum, Portulaca oleracea (purslane) extract, sodium hyaluronate, tocopherol, phenoxyethanol, biotin, Argania spinosa kernel oil, hydroxyethylcellulose, xanthan gum, carbomer, and triethanolamine

Typically, 10 ml is used for each application. An alternative product: Serum Hydro Tens FGF+supplied by Pure Code Co., Ltd. Website is https://purecode.co.jp/cosmetics.html#l_cosme03 and includes as ingredients: water, butylene glycol, glycerin, 1,2-hexanediol, caprylyl glycol, glycosyl trehalose, hydrogenated starch hydrolysate, ascorbyl glucoside, sh-oligopeptide-13, acetyl hexapeptide-8, ergothioneine, palmitoyl tetrapeptide-7, palmitoyl oligopeptide, dipotassium glycyrrhizate, Scutellaria baicalensis extract, sodium hyaluronate, 6-aminocaproic acid, carbomer, polysorbate 20, sodium citrate, citric acid, and HEDA-4Na.

In performing the two steps of this first process, the application of the therapeutic effective hair growth serum should be done promptly after the micro-needling process. Typically, the first process should be conducted only once a month, as the micro-needling causes minor punctures in the treated area that may need time to recover for better results.

II. Second Process Involves a Transmission of Electric Current to the Dermis of the Area to be Treated.

The first step in the second process is the transmittal of low electric current to the dermis of the area to be treated for approximately 30 minutes per session. This electric current awakens the nerves at the surface of the area to be treated to increase blood flow to the area and activate the adjacent cells to allow the applied nutrients to reach the surface of the area to be treated to promote hair growth. The level of electric current is low and the person being treated may often not even notice the electric current being applied, but the current level needs to be sufficient and for a duration sufficient for the person receiving the treatment to feel eventually a stimulating electric sensation in the scalp, or the area to be treated. It is vital to repeat the application of the electric current until the person receiving the treatment feels a stimulating electric sensation.

A suitable product used for the application of this electric current is the BeBe Pinocchio DM-5 SUPER DX Plus supplied by Pure Code Co., Ltd. of Japan. Website: https://purecode.co.jp/pinocchio.html

The second step of Process II is the application of hair growth therapeutic(s). Upon completion of transmittal of low electric current to the dermis of the area to be treated, one or more of the following hair growth therapeutic(s) products in a therapeutic effective amount as determined by trial and error, depending upon the results obtained and desired, should be applied to the area to be treated to promote hair growth. Step One, which is commonly known as “cytoplasm system,” creates temporary micro holes in the dermis which allows hair growth products to be better absorbed. The determination of the product to be used is based on the results obtained which are dependent upon the hair type of the person being treated and desired results.

One product to be used is SerumActive SA supplied by Pure Code Co., Ltd. of Japan. Website: https://purecode.co.jp/cosmetics.html#l_cosme03

The ingredients include water, placental extract, propanediol, glycerin, pentylene glycol, sodium hyaluronate, hydrolyzed hyaluronic acid, hydrolyzed collagen, soluble proteoglycan, Passiflora edulis fruit extract, cerebrosides, arginine, dipotassium glycyrrhizate, butylene glycol, calcium sodium borosilicate, silver oxide, Salix alba (willow) bark extract, Citrus grandis (grapefruit) seed extract, carbomer, xanthan gum, ethanol.

A second acceptable product is GrandeBROW Brow Enhancing Serum supplied by Grande Cosmetics LLC, Website: https://grandecosmetics.com/collections/all/products/grandebrow which includes water/aqua/eau, panthenol, glycerin, sodium citrate, leuconostoc/radish root ferment filtrate, alanine, alcohol, aloe barbadensis leaf juice, arctostaphylos uva ursi leaf extract, arginine, aspartic acid, calcium gluconate, caprylyl glycol, flower extract, ethylhexylglycerin, gluconolactone, glycine, hexyl glycol, histidine, honey extract/mel extract/extrait de miel, hydrolyzed glycosaminoglycan, hydrolyzed yeast extract, hydroxyethylcellulose, isoleucine, isopropyl cloprostenate, magnesium ascorbate, magnesium phosphate, polysorbate 20, proline, propylene glycol, serine, sodium hyaluronate, sodium lactate, sodium pca, threonine, tocopheryl acetate, triethanolamine, Tussilago farfara (coltsfoot) leaf extract, valine, Vitis vinifera (grape) seed extract, phenoxyethanol, sodium benzoate, sodium metabisulfite.

A third product that can be used is GrandeHAIR Enhancing Serum supplied by Grande Cosmetics LLC having a website: https://grandecosmetics.com/products/grandehair-rejuvenation-serum and includes as ingredients: water/aqua/eau, glycerin, butylene glycol, polysorbate 20, peg-40 hydrogenated caster oil, menthyl lactate, alanine, alcohol, apigenin, arctostaphylos uva ursi leaf extract, arginine, aspartic acid, biotinoyl tripeptide-1, bisabolol, Calendula officinalis flower extract, Camellia oleifera leaf extract, Carica papaya (papaya) fruit extract, disodium edta, Geranium maculatum extract, Ginkgo biloba leaf extract, glycine, glycine, Glycyrrhiza glabra (licorice) root extract, histidine, hydrolyzed wheat protein, isoleucine, isopropyl cloprostenate, magnesium ascorbylphosphate, oleanolic acid, Panax ginseng root extract, panthenol, fragrance/parfum, pca, phenylalanine, Pilocarpus pennatifolius leaf extract, ppg buteth-26, proline, serine, sodium lactate, sodium pca, Taraxacum officinale, dandelion extract, threonine, valine, Vitis vinifera (grape) seed extract, benzoic acid, chlorphenesin, phenoxyethanol, sodium benzoate, sodium metabisulfite, sorbic acid, alpha-isomethyl ionone, benzyl benzoate, butylphenyl methylpropional, citronellol, hexylcinnamaldehyde, hydroxyisohexyl 3-cyclohexene carboxaldehyde.

A fourth product found useful is Scalp D beaute Pure Free Eyelash supplied by Angfa Co., Ltd. of Japan, website: https://www.angfa-store.jp/product/BTPFP000A. This product includes as ingredients: platinum (for skin conditioner), biotinoyl tripeptide-1, panthenol, glycerin, pyrrolidinyl diaminopyrimidine oxide (all for humectants), cystine, glutamic acid (all for lifting and tightening).

III. The Third Process Involves Use of an Electric Brush for a Massagingtreatment of the Area.

A preferred product is the Electric Bari Brush supplied by GM Corporation of Japan, Website: https://shop.el.gm-beauty.jp/shopdetail/000000000017/

The silver pinheads at the very tip of the Electric Bari Brush emit a low frequency which vibrates the arrector pili muscle that attaches to the base of a hair follicle at one end and to dermal tissue on the other end at a frequency more than 1,000 times per second. As a result, the electric brush removes stiffness and waste present in the thin fascia covering the scalp, or area to be treated. By softening the area to be treated, hair growth is promoted. Preferably, the electric brush should be used continuously for 19 minutes. The electric brush should be used on the areas to be treated for the entire duration. If the area to be treated is dry, some discomfort may be felt and water or toner may be applied to moisturize the surface of the treated area to lessen discomfort.

It is considered important to utilize Process I once a month, but Process II and Process III should be conducted at least twice a week (preferably everyday) in between applications of Process I. After a month, if hair regrowth is evident, that is some lanugo appears, only Process II and Process III need to be repeated. If lanugo does not appear after a month of application of Process I, Process I should be repeated monthly, along with Process II and Process III.

What is claimed is:

1. A method for treatment for rejuvenation of hair growth combining three phases including a first phase of micro-needling the area to be treated and thereafter applying an effective amount of a therapeutic; a second phase of applying an electric current and thereafter applying an effective amount of a therapeutic; and a third phase of a mechanical massage.

2. A method for treatment for rejuvenation of hair growth as set forth in claim 1, applying said first phase at least once per month until satisfactory results are achieved.

3. A method for treatment for rejuvenation of hair growth as set forth in claim 2, applying said second and third phases twice a week.
